(19) United States
(12) Patent Application Publication
(10) Pub. No.: US 2014/0109906 A1
(43) Pub. Date: Apr. 24, 2014

(54) AEROSOL MASK WITH SWIVEL ADAPTER

(71) Applicants: Anthony Wells, Lufkin, TX (US); Deidra Cross, Lufkin, TX (US)

(72) Inventors: Anthony Wells, Lufkin, TX (US); Deidra Cross, Lufkin, TX (US)

(21) Appl. No.: 13/659,391

(22) Filed: Oct. 24, 2012

(51) Int. Cl. A61M 16/08; A61M 16/14; A61M 11/06; A61M 16/06

(52) U.S. Cl. CPC A61M 16/0816; A61M 16/0683; A61M 16/14; A61M 11/06
USPC 128/203.29

(57) ABSTRACT

An aerosol mask may include a mask body, having a top portion of the mask body cut short to prevent the intrusion of the aerosol mask into the eyes of a patient, a bottom portion of the mask body being adapted to fit under the chin of the patient; a swivel adapter attached to the center of the aerosol mask; and an elbow member coupled to the swivel adapter.

### PRIORITY

The present invention claims priority under 35 USC section 119 based upon a provisional application which was filed on Oct. 24, 2011 with a Ser. No. 61/550,839.

### FIELD OF THE INVENTION

The present invention relates generally to connecting systems for respiratory therapy apparatus, and specifically to such a connecting system designed to connect a respiratory connection device to a mask device, the mask device being secured to the patient.

### BACKGROUND

During continuous mechanical ventilation/respiration of patients, a ventilation tube delivers air or oxygen to the patient from a remote source. The ventilation system often includes a ‘Y’-piece having inhalation and exhalation sockets for connection to appropriate tubes, and a third socket for connection to the ventilation tube. The end of the ventilation tube is inserted into the patient's nostril (nasal endotracheal tube), into the patient's mouth (oral endotracheal tube), into a face mask releasably secured to the patient, or into an incision made in the patient's neck (endotracheal or tracheostomy tube) so that the tube may be inserted directly into the trachea. If the patient moves slightly, for example by moving his head, the tube may rub against sensitive surfaces inside the patient's respiratory tract, causing pain and/or injury.

Conventional respiratory connectors have addressed this problem by providing swiveling connectors between the ‘Y’-piece and the patient. One disadvantage of such conventional connectors is that they are relatively easily dislodged from the ventilation tube and/or the patient connection, either by the patient or medical personnel. Since any disruption of the respiratory therapy may be fatal, or at a minimum, critical to the patient's condition, this disadvantage takes on added significance.

Another disadvantage of conventional connectors is that they form an ineffective seal which permits leakage of respiratory gases, often containing aerosolized medication or bacterial and/or viral particles. Aside from affecting the efficiency of treatment and the safety of the patient, this latter disadvantage may affect the health of attending physicians and/or health care workers.

Thus, there is a need for a connector for a respiratory therapy apparatus which is not easily disconnected from the ventilation tube and/or the patient, and which forms a gas-tight seal in the swivel area.

U.S. Pat. No. 5,062,420 discloses a sealed swivel for a respiratory apparatus including a first end configured for engagement with either an endotracheal tube or a face mask, a second end with a telescoping formation for insertion into a tubular respiratory connector, and a central portion with a sealing formation defining a swivel track and configured for creating a gas tight seal while permitting relatively free swiveling action.

U.S. Pat. No. 7,082,947 discloses face masks for use in pressurized drug delivery applications, such as aerosol drug delivery systems, and a method of reducing aerosol deposition in the region of the eyes are presented. The face masks according to the various embodiments disclosed herein contain features that reduce the inertia of the aerosolized drug in perinasal areas. This results in a reduction in the amount of aerosolized drug that is deposited in the region of the eyes by inertial impaction, while at the same time, the features are constructed to maintain the flow of the aerosolized drug into the face mask so that the aerosolized drug is effectively delivered to the respiratory system of the patient.

U.S. Pat. No. 8,042,540 discloses a mask for delivery of oxygen to a patient, including a body having a peripheral portion, a central portion, and bridge portions extending between the central portion and the peripheral portion. The central portion has an inner surface and an outer surface, the inner surface to be oriented towards the patient's face when the mask is in position, the inner surface of the central portion being provided with a wall circumscribing a base. The wall and base circumscribe a centrally positioned oxygen delivery aperture or oxygen diffuser to direct the flow of oxygen generally towards the patient's nose and mouth when the mask is in use. An element associated with the aperture and the central portion releasably receives and secures in position an oxygen delivery tube.

US patent application 20030127101 discloses respiratory mask being reduced in size to better fit patients with smaller faces by either (a) folding one or more accordion folds formed in an upper portion of the mask, or (b) tearing away a part of the upper portion of the mask. The respiratory mask for adult patients can thus be adjusted to fit smaller adults and larger children. The respiratory mask for children can thus be adjusted to fit smaller children. A ball joint swivel connector is provided to connect the gas inlet of the masks to the hose connection for respiratory or nebulizer treating gases or aerosols.

US patent application 20100000538 discloses a mask assembly for use in providing a supply of air at positive pressure to the airways of a patient includes a frame and an elbow. The elbow is rotatable with respect to the frame when assembled. The frame includes an elbow-receiving portion. The elbow and elbow-receiving portion of the frame are adapted to include respective interlocking sealing portions and one of the elbow and elbow-receiving portion includes a flexible element that upon assembly flexes to introduce a preload that effects a seal between the respective interlocking sealing portions.

### SUMMARY

An aerosol mask may include a mask body, having a top portion of the mask body cut short to prevent the intrusion of the aerosol mask into the eyes of a patient, a bottom portion of the mask body being adapted to fit under the chin of the patient; a swivel adapter attached to the center of the aerosol mask; and an elbow member coupled to the swivel adapter.

The swivel adapter may rotate a complete 360°.

The elbow may include a first retaining lip.

The elbow may include a second retaining lip.

The second retaining lip may include a surface an acute angle.

The second retaining lip may be deformable.

### BRIEF DESCRIPTION OF THE DRAWINGS

The invention may be understood by reference to the following description taken in conjunction with the accompanying drawings, in which, like reference numerals identify like elements, and in which:

FIG. 1 is a side view of an aerosol mask with a swivel adapter according to an embodiment of the present invention;

FIG. 2 is a perspective view of the aerosol mask with the swivel adapter;

FIG. 3 illustrates an exploded view of aerosol mask with the swivel adapter of the present invention;

FIG. 4 illustrates a connection ring of the swivel adapter of the present invention;

FIG. 5 illustrates a perspective view of the elbow of the present invention;

FIG. 6 illustrates a cross-sectional view of the elbow of the present invention.

### DETAILED DESCRIPTION

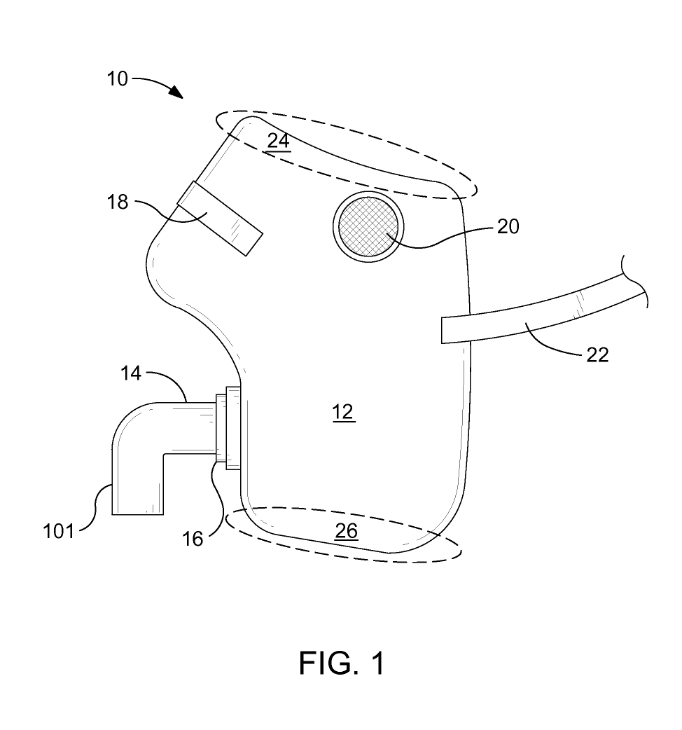

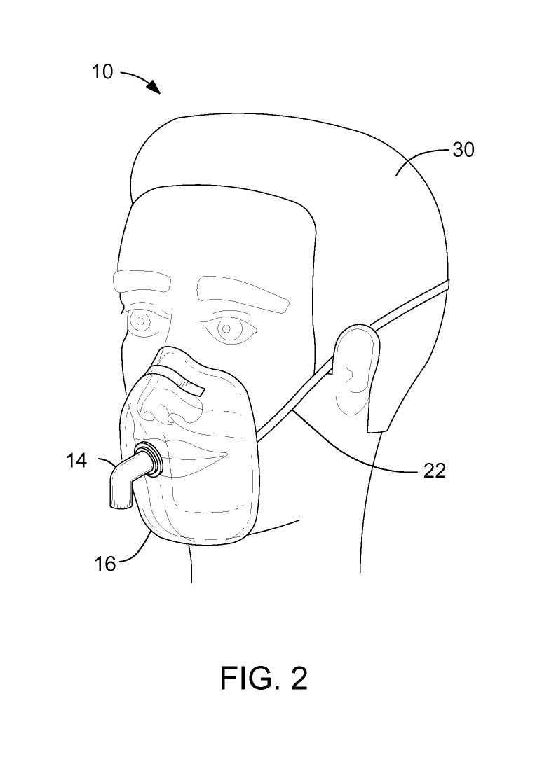

The following detailed description is of the currently contemplated modes of carrying out exemplary embodiments of the invention. The description is not to be taken in a limiting sense, but is made merely for the purpose of illustrating the general principles of the invention, since the scope of the invention is best defined by the appended claims. Broadly, the present invention generally provides a connection system 100 to cooperate with an aerosol mask 12 with a swivel adapter 101 which may include a connection ring 16 for a friction fit with the aerosol mask 12 and a respiratory connection device 14 for attaching a nebulizer or venturi device. Referring now to FIGS. 1 and 2, different views of an aerosol mask 10 with a swivel adapter 101 are shown according to an exemplary embodiment of the present invention. The aerosol mask 10 may include a mask body 12. The mask body 12 may be made of a soft pliable non-latex, plastic material. The mask body 12 may have a semi-oval shape. A top portion 24 of the mask 10 may be may be formed short to prevent the intrusion of the mask into the eyes of a patient 30. The bottom portion 26 of the mask 10 may be made longer than a regular medical mask to fit under the chin of the patient. The aerosol mask 10 may provide a better fit to the patient's face that may result in more comfort and assure a better seal. An elbow member 14 which may be a L-shaped tube which may have a central aperture may be attached via a 360-degree swivel adapter 16 which may be a ring with a center aperture 103 to the center of the aerosol mask 10 near a location of the mouth of the patient 30. A nebulizer (not shown) may be fitted inside the elbow member 14. An aerosol delivery tube (not shown) may also be fitted on an open end of the elbow member 14 to deliver medication to the patient 30. A flexible thin metal strip 18 may be affixed on the outside of the mask body 12 to be shaped over the bridge of the nose area of the patient 30. An elastic strap 22 may be attached to the mask body 12 for holding the aerosol mask 10 onto the face of the patient 30. A pair of exhalation ports 20 may be formed in the mask body 12 close to the nose of the patient 30.

The disclosed aerosol mask 10 with the free moving 360-degree swivel adapter 14 which may rotate clockwise and counterclockwise and which may rotate a full 360° without reversing may provide near optimal delivery of medication in any position, while it may be comfortably and securely fitted on the face of the patient 30.

FIG. 3 discloses an elbow member 14 which may be a L-shaped tube which may have a central aperture may be attached via a 360-degree swivel adapter 16 which may be a ring with a center aperture 103 to the center of the aerosol mask 10 near a location of the mouth of the patient 30. The mask body 12 may be made of a soft pliable non-latex, plastic material. The mask body 12 may have a semi-oval shape. A top portion 24 of the mask 10 may be may be cut short to prevent the intrusion of the mask into the eyes of a patient 30. The bottom portion 26 of the mask 10 may be made longer than a regular medical mask to fit under the chin of the patient. The aerosol mask 10 may provide a better fit to the patient's face that may result in more comfort and assure a better seal. An elbow member 14 which may be a L-shaped tube which may have a central aperture may be attached via a 360-degree swivel adapter 16 which may be a ring with a center aperture 103 to the center of the aerosol mask 10 near a location of the mouth of the patient 30.

FIG. 3 additionally illustrates a mask aperture 105 to cooperate with the swivel adapter 16, and more particularly, the elbow 14 cooperates with the outer perimeter of the mask aperture 105.

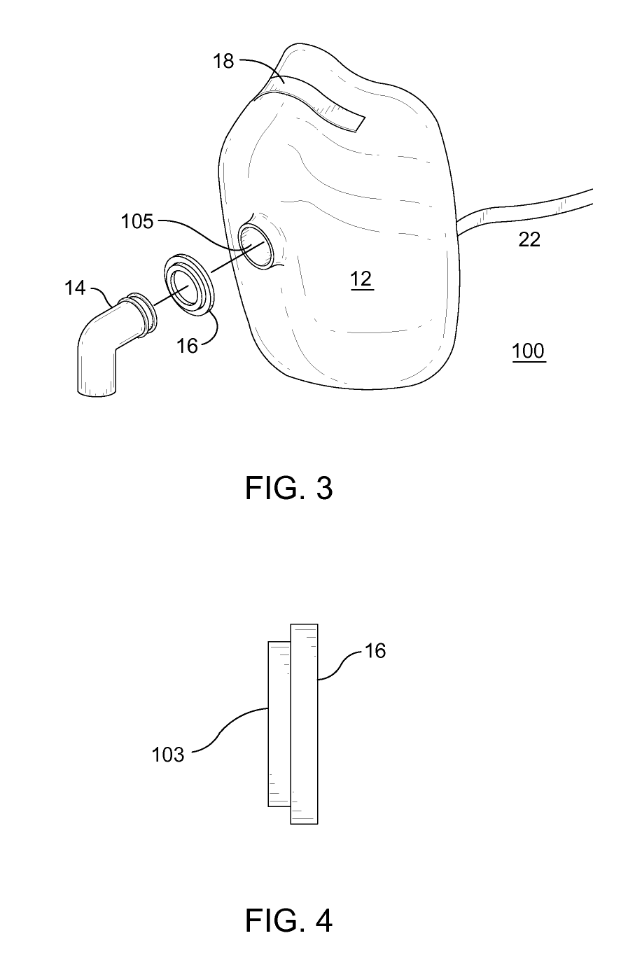

FIG. 4 illustrates a side view of the connection ring 16 and illustrates the central aperture 103.

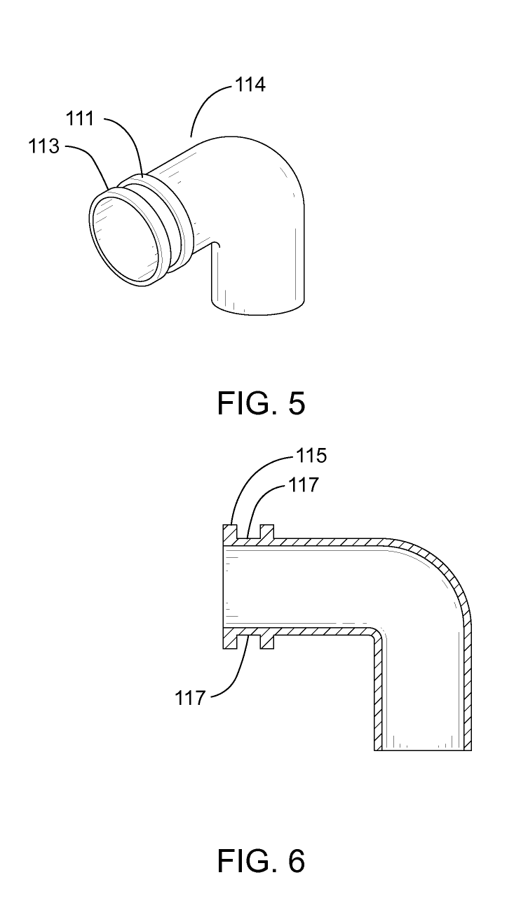

FIG. 5 illustrates a perspective view of the elbow 14 which may include a first retaining lip 111 which may extend along the outer periphery of the elbow 14 and a second retaining lip 113 which may also extend along the outer periphery of the elbow 14 and may be positioned at one end of the elbow 14, and an outer surface 115 of the second retaining lip 113 may be at an acute angle alpha α. The retaining lip 113 may be deformable and flexible (nondestructive) to allow perimeter of the aperture 105 to be positioned between the first retaining lip 113 and the second retaining lip 115 in the channel 117 defined by the first retaining lip 113 and the second retaining lip 115.

It should be understood, of course, that the foregoing relates to exemplary embodiments of the invention and that modifications may be made without departing from the spirit and scope of the invention as set forth in the attached claims

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1) An aerosol mask, comprising:
a mask body, wherein
a top portion of the mask body cut short to prevent the intrusion of the aerosol mask into the eyes of a patient,
a bottom portion of the mask body is adapted to fit under the chin of the patient;
a swivel adapter attached to the center of the aerosol mask; and
an elbow member coupled the swivel adapter.

2) An aerosol mask as in claim 1, wherein the swivel adapter rotates a complete 360°.

3) An aerosol mask as in claim 1, wherein the elbow includes a first retaining lip.

4) An aerosol mask as in claim 1, wherein the elbow includes a second retaining lip.

5) An aerosol mask as in claim 4, wherein the second retaining lip includes a surface an acute angle.

6) An aerosol mask as in claim 4, wherein the second retaining lip is deformable.
